(19) United States
(12) Patent Application Publication
(10) Pub. No.: US 2026/0240509 A1
(43) Pub. Date: Aug. 20, 2026

(54) MEDICAL IMAGE PROCESSING APPARATUS, POSITRON EMISSION TOMOGRAPHY (PET) APPARATUS, AND MEDICAL IMAGE PROCESSING METHOD

(71) Applicant: CANON MEDICAL SYSTEMS CORPORATION, Otawara-shi (JP)

(72) Inventor: Kenta MORIYASU, Nasushiobara (JP)

(73) Assignee: CANON MEDICAL SYSTEMS CORPORATION, Otawara-shi (JP)

(21) Appl. No.: 19/540,066

(22) Filed: Feb. 13, 2026

(30) Foreign Application Priority Data
Feb. 17, 2025 (JP) 2025-023481

(51) Int. Cl. A61B 6/03; A61B 6/00

(52) U.S. Cl. CPC A61B 6/032; A61B 6/4417

(57) ABSTRACT

A medical image processing apparatus of an embodiment is configured to process scan data acquired by a plurality of detectors detecting gamma ray emitted from a subject mounted on a couchtop. The medical image processing apparatus includes processing circuitry. The processing circuitry is configured to acquire a first plurality of scan data corresponding to a first region of the subject in a longitudinal direction of the couchtop and a second plurality of scan data corresponding to a second region of the subject in the longitudinal direction, estimate scan data corresponding to a region of the subject different from the first region in a first period based on the first plurality of scan data and the second plurality of scan data, and generate a PET image corresponding to the region of the subject different from the first region in the first period based on the estimated scan data.

### CROSS-REFERENCE TO RELATED APPLICATIONS

This application is based upon and claims the benefit of priority from Japanese Patent Application No. 2025-023481, filed February 17, 2025, the entire contents of which are incorporated herein by reference.

### FIELD

Embodiments described in the present specification and drawings relate to a medical image processing apparatus, a positron emission tomography (PET) apparatus, and a medical image processing method.

### BACKGROUND

In recent years, apparatuses that perform total body positron emission tomography (PET) (hereinbelow, referred to as "total body PET apparatuses"), which are capable of scanning a whole body of a subject at once, have become known. A total body PET apparatus is equipped with a PET detector, which is long enough to cover a whole body of a subject along a body axis direction. Accordingly, unlike conventional PET apparatuses, the total body PET apparatus is capable of observing time series images (whole body dynamic images) of a process from drug administration to distribution throughout a subject body.

### BRIEF DESCRIPTION OF THE DRAWINGS

FIG. 1 is a configuration diagram illustrating an example of a positron emission tomography (PET) apparatus according to an embodiment;

FIG. 2A is a diagram illustrating images captured by a total body PET apparatus;

FIG. 2B is a diagram illustrating a relationship among whole body images captured by the total body PET apparatus;

FIG. 3A is a diagram illustrating time series transition of a scan range by the PET apparatus according to the embodiment;

FIG. 3B is a diagram illustrating whole body image data (relationship between an actual scan range and an estimation range) according to the embodiment;

FIG. 4 is a flowchart illustrating an example of processing for generating a whole body image by the PET apparatus according to the embodiment;

FIG. 5A is a diagram illustrating a flow of scan processing that is executed by the PET apparatus according to the embodiment;

FIG. 5B is a diagram illustrating input and output data of a trained model according to the embodiment;

FIG. 6A is a diagram illustrating training data prepared based on whole body scan data captured by the total body PET apparatus; and

FIG. 6B is a diagram illustrating input and output data of the trained model in a training phase according to the embodiment.

### DETAILED DESCRIPTION

A medical image processing apparatus is configured to process scan data acquired by a plurality of detectors detecting gamma ray emitted from a subject mounted on a couchtop, the plurality of detectors being supported by a frame included in a positron emission tomography (PET) apparatus, at least one of the couchtop or the frame being configured to move in a longitudinal direction of the couchtop. The medical image processing apparatus includes processing circuitry. The processing circuitry is configured to: acquire a first plurality of scan data corresponding to a first region of the subject in the longitudinal direction and a second plurality of scan data corresponding to a second region of the subject in the longitudinal direction, the first plurality of scan data being acquired in a first period by the plurality of detectors in a state where the plurality of detectors is located at a position corresponding to the first region, the second plurality of scan data being acquired in a second period after the first period by the plurality of detectors in a state where the plurality of detectors is located at a position corresponding to the second region; estimate scan data corresponding to a region of the subject different from the first region in the first period based on the first plurality of scan data and the second plurality of scan data; and generate a PET image corresponding to the region of the subject different from the first region in the first period based on the estimated scan data.

Various embodiments will be described hereinafter with reference to the accompanying drawings.

A medical image processing apparatus, a PET apparatus, and a medical image processing method according to the embodiments are described below with reference to the accompanying drawings. The medical image processing apparatus is, for example, a PET apparatus or a PET computed tomography (PET-CT) apparatus. In the following description, a case in which the medical image processing apparatus is a PET apparatus is used as an example.

### First Embodiment

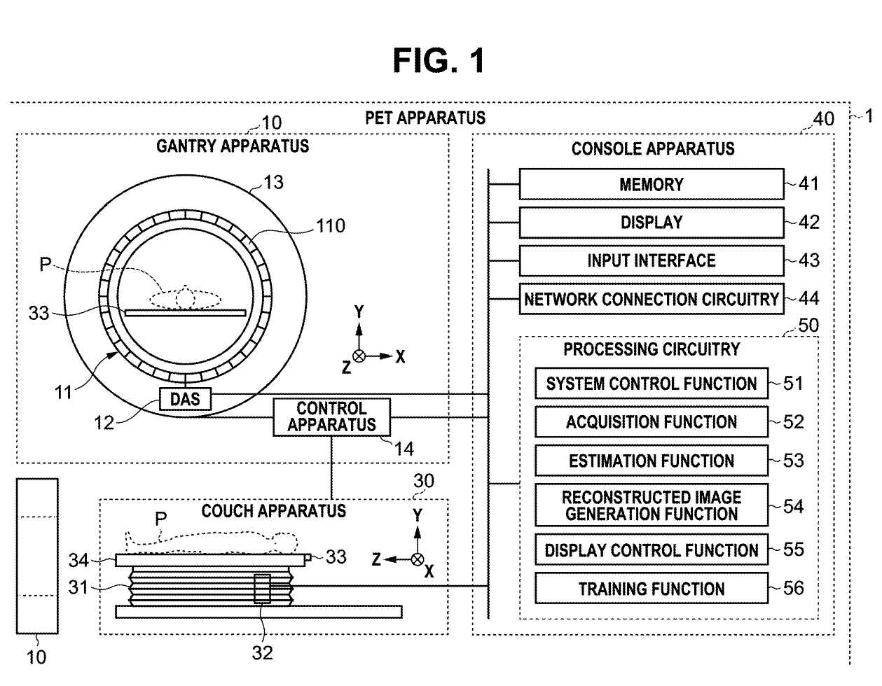

FIG. 1 is a configuration diagram of a PET apparatus 1 according to a first embodiment. The PET apparatus 1 is a medical diagnostic apparatus that detects radiation emitted by a radioactive substance contained in a radiopharmaceutical administered to a subject P and, from the detected radiation dose, determines whether and where the radiopharmaceutical accumulates. The PET apparatus 1 generates and displays an image corresponding to the detected radiation dose (hereinbelow, also referred to as "PET image"). Accordingly, a person who performs a PET examination (a doctor, a technician, or the like) can visually confirm whether the subject P has a lesion.

The PET apparatus 1 uses a detector that cannot cover a whole body of a subject, repeatedly collects partial images of the subject in a short period of time, and generates information corresponding to total body PET (for example, time series whole body dynamic images, hereinbelow, simply referred to as "whole body image") based on the plurality of collected images. Thus, the PET apparatus 1 can acquire whole body images equivalent to those of the total body PET, for example, from drug administration to distribution throughout the subject body even without using a detector that can cover the whole body of the subject.

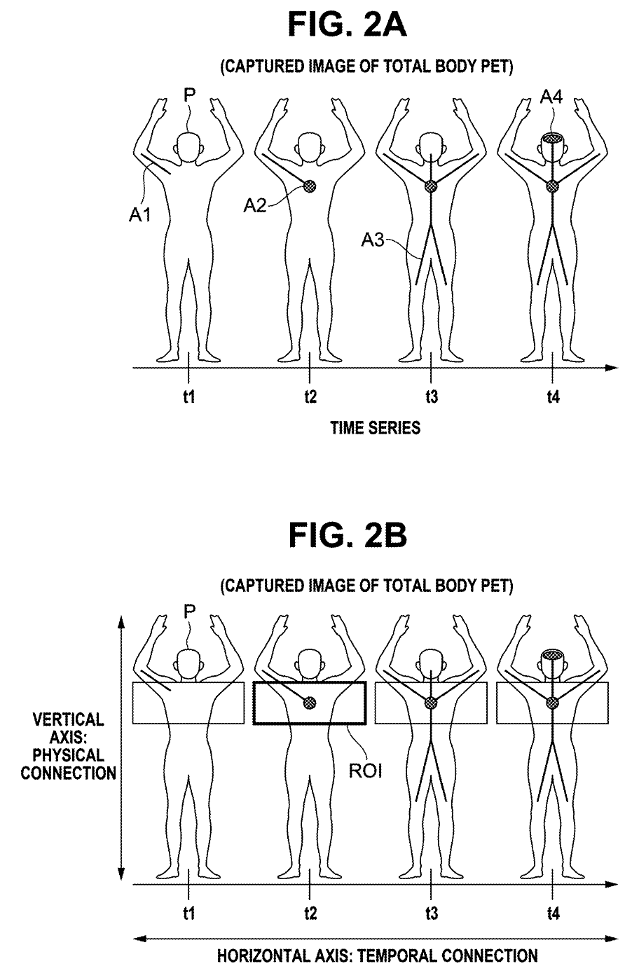

In order to generate a whole body image based on data acquired by the detector that cannot cover the whole body, it is important to determine how to acquire missing data. FIG. 2A is a diagram illustrating images captured by a total body PET apparatus equipped with a detector wide enough (long enough) to cover a whole body. The total body PET apparatus can capture an image of the whole body of the subject P at once. In the example illustrated in FIG. 2A, a first whole body image is captured at time t1 when a drug is administered into a vein of an arm region A1 of the subject P. A second whole body image is captured at time t2 when the drug reaches a heart region A2. A third whole body image is captured at time t3 when the drug further spreads throughout the whole body and reaches a leg region A3. A fourth whole body image is captured at time t4 when the drug reaches a head region A4. In this way, imaging using the total body PET apparatus can acquire time series whole body images of the process from drug administration to distribution throughout the whole body of the subject P.

FIG. 2B is a diagram illustrating a relationship among whole body images captured by the total body PET apparatus. In FIG. 2B, when focusing on a specific region within the whole body image (for example, a region including the heart, hereinbelow referred to as a "region of interest (ROI)"), there is a temporal correlation in data in a horizontal axis (time axis) direction between the whole body images, and there is also a physiological correlation in data in a vertical axis (body axis) direction due to blood flow within the body parts. In other words, there is data correlation between the whole body images in the horizontal axis (time axis) direction and the vertical axis (body axis) direction. The PET apparatus 1 according to the present embodiment focuses on such data correlation and performs processing for estimating missing data in each scan.

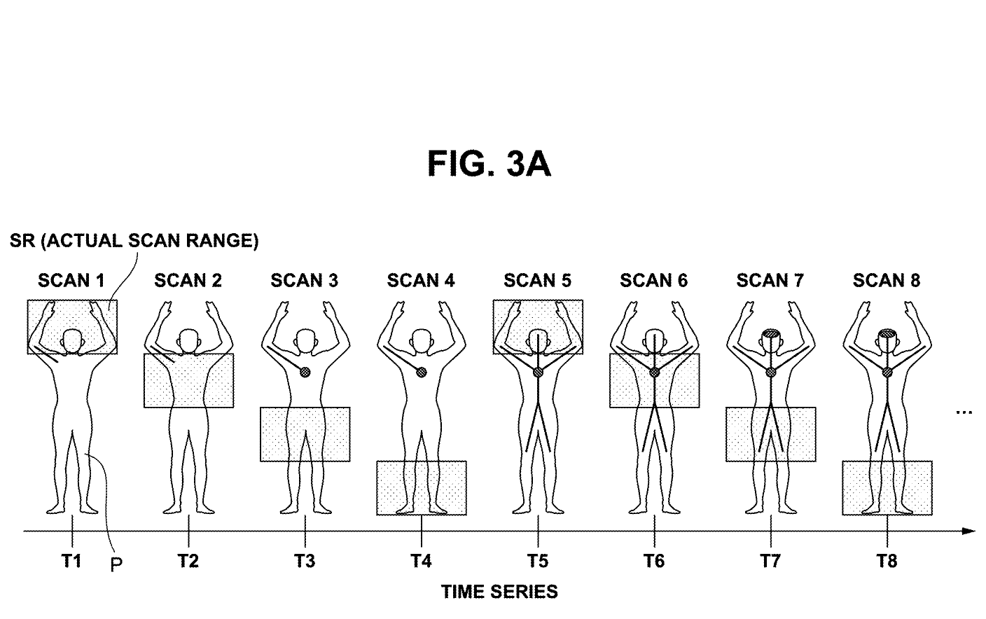

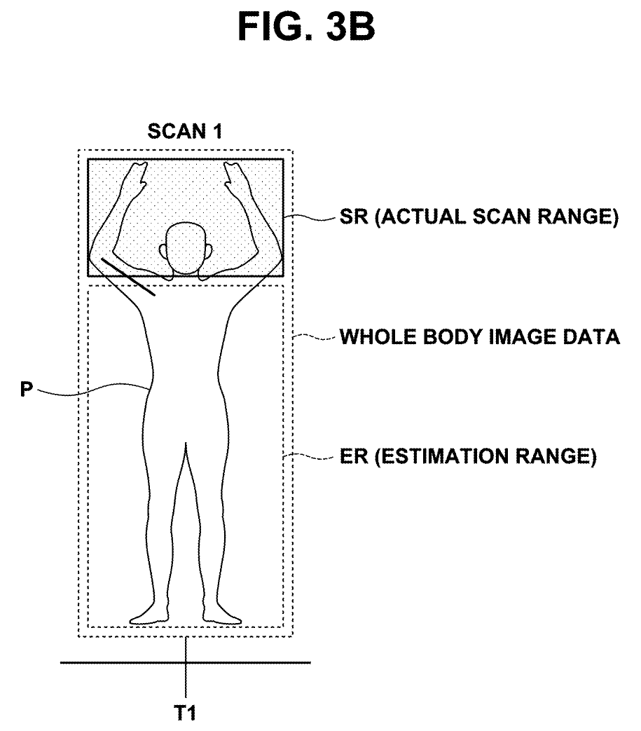

FIG. 3A is a diagram illustrating time series transition of a scan range by the PET apparatus 1 according to the embodiment. As illustrated in FIG. 3A, the PET apparatus 1 equipped with the detector that cannot cover the whole body, for example, repeatedly performs scans (data collection) from the head region to the leg region of the subject P in the body axis direction. For example, when an actual scan range of the PET apparatus 1 is indicated as an actual scan range SR, the head region of the subject P is scanned in a scan 1 (time T1), a chest region of the subject P is scanned in a scan 2 (time T2), a lower abdomen region of the subject P is scanned in a scan 3 (time T3), the leg region of the subject P is scanned in a scan 4 (time T4), and then scanning is repeated in a similar manner for each body region of the subject P. Based on the scan data acquired in this way for each body region of the subject P, missing data in each scan is estimated, and the whole body image is generated for each scan.

FIG. 3B is a diagram illustrating whole body image data (relationship between the actual scan range and the estimation range) according to the embodiment. As illustrated in FIG. 3B, the head region of the subject P is the actual scan range SR in the scan 1 (time T1). In this case, data in an estimation range ER, which is the missing data in the scan 1 (data other than the head region), is inferred, and the whole body image including the actual scan range SR and the estimation range ER in the scan 1 is generated. Here, since the drug distribution changes moment by moment, a time for each scan (scan interval) is set shorter than those in conventional methods.

### Configuration of PET Apparatus

Returning to FIG. 1, the PET apparatus 1 includes, for example, a gantry apparatus 10, a couch apparatus 30, and a console apparatus 40. While, for convenience of explanation, FIG. 1 illustrates both a view of the gantry apparatus 10 in a Z-axis direction and a view in an X-axis direction, in reality, the PET apparatus 1 is provided with one gantry apparatus 10. According to the present embodiment, a central axis of a frame 13 in a non-tilted state or a longitudinal direction of a couchtop 33 of the couch apparatus 30 is defined as the Z-axis direction, an axis perpendicular to the Z-axis direction and horizontal to a floor surface is defined as the X-axis direction, and a direction perpendicular to the Z-axis direction and vertical to the floor surface is defined as a Y-axis direction. The PET apparatus 1 and/or the console apparatus 40 are an example of a "medical image processing apparatus" as described in the claims.

The gantry apparatus 10 includes, for example, a radiation detector 11, a data acquisition system (hereinbelow, referred to as "DAS") 12, the frame 13, and a control apparatus 14.

The radiation detector 11 detects radiation, such as gamma rays, emitted from the subject P (more specifically, from a radiopharmaceutical administered to the subject P). The radiation detector 11 outputs an electrical signal (which may be an optical signal or the like) corresponding to an amount of detected radiation (radiation dose) to the DAS 12. The radiation detector 11 has a cylindrical shape and is disposed to surround a periphery of an imaging port of the gantry apparatus 10. The radiation detector 11 includes a plurality of PET detection elements 110 that are arranged, for example, in circumferential and central axis directions. Each of the PET detection elements 110 detects radiation emitted to the surroundings from the subject P placed within the imaging port. The radiation detector 11 outputs an electrical signal corresponding to the radiation dose detected by each of the PET detection elements 110 to the DAS 12. The radiation detector 11 is an example of a "detector" as described in the claims.

The DAS 12 includes, for example, an amplifier, an integrator, and an analog-to-digital (A/D) converter. The amplifier amplifies an electrical signal output from each of the PET detection elements 110 of the radiation detector 11. The integrator integrates the amplified electrical signal over a predetermined time interval. The A/D converter converts the electrical signal indicating the integration result by the integrator into a digital signal. The DAS 12 outputs acquired data based on the digital signal to the console apparatus 40. The acquired data is, for example, a digital value indicating the radiation dose that has been acquired at each position within the radiation detector 11 by the PET detection elements 110 arranged therein.

The frame 13 is an annular member that supports the radiation detector 11 and the DAS 12. The frame 13 is not limited to an annular member and may be an arm-like member as long as the frame 13 can support the radiation detector 11 and the DAS 12.

For example, in a case where a medical information processing apparatus according to the present embodiment is a PET-CT apparatus, in addition to the radiation detector 11 and the DAS 12, the frame 13 may also support a rotation frame in which an X-ray tube that generates X-rays to be irradiated onto the subject P and an X-ray detector that detects intensity of the X-rays having passed through the subject P and entering therein are fixed in positions facing each other so that the rotation frame is supported to be rotatable freely around the subject P introduced inside.

The control apparatus 14 is provided with, for example, processing circuitry that includes a processor, such as a central processing unit (CPU) or the like. The control apparatus 14 receives an input signal from an input interface 43 attached to the console apparatus 40 or the gantry apparatus 10 and controls operations of the gantry apparatus 10 and the couch apparatus 30. The control apparatus 14, for example, tilts the gantry apparatus 10 and moves the couchtop 33 of the couch apparatus 30. When tilting the gantry apparatus 10, the control apparatus 14 tilts the frame 13 around an axis parallel to the Z-axis direction based on a tilt angle input to the input interface 43. The control apparatus 14 recognizes the tilt angle of the frame 13 based on an output from a sensor (not illustrated). Further, the control apparatus 14 provides the tilt angle of the frame 13 to a processing circuitry 50 as needed. The control apparatus 14 may be provided in the gantry apparatus 10 or in the console apparatus 40. For example, in a case where a medical information processing apparatus is a PET-CT apparatus, the control apparatus 14 may be provided with a drive mechanism including, for example, a motor, an actuator, and the like, which rotationally operates the rotation frame included in the frame 13.

The couch apparatus 30 is an apparatus that places and transports the subject P to be scanned, and introduces the subject P into the frame 13 of the gantry apparatus 10. The couch apparatus 30 includes, for example, a base 31, a couch driving apparatus 32, the couchtop 33, and a support frame 34. The base 31 includes a housing that movably supports the support frame 34 in a vertical direction (Y-axis direction). The couch driving apparatus 32 includes a motor and an actuator. The couch driving apparatus 32 moves the couchtop 33 on which the subject P is placed in the longitudinal direction (Z-axis direction) of the couchtop 33 along the support frame 34. The couchtop 33 is a plate-like member on which the subject P is placed.

The couch driving apparatus 32 may move not only the couchtop 33 but also the support frame 34 in the longitudinal direction of the couchtop 33. Conversely to the above-described configuration, the gantry apparatus 10 may be movable in the Z-axis direction, and the frame 13 may be controlled to come around the subject P by the movement of the gantry apparatus 10. Both the gantry apparatus 10 and the couchtop 33 may be configured to be movable. Further, the PET apparatus 1 may be a type of apparatus that scans the subject P in a standing or sitting position. In this case, the PET apparatus 1 includes a subject support mechanism instead of the couch apparatus 30, and the gantry apparatus 10 moves the frame 13 in a direction vertical to the floor surface.

The console apparatus 40 includes, for example, a memory 41, a display 42, the input interface 43, network connection circuitry 44, and the processing circuitry 50. According to the present embodiment, the console apparatus 40 is described as being separate from the gantry apparatus 10, but the gantry apparatus 10 may include part or all of the components of the console apparatus 40.

The memory 41 is realized by, for example, a read only memory (ROM), a random access memory (RAM), a semiconductor memory element, such as a flash memory, a hard disk drive (HDD), an optical disk, and the like. The memory 41 stores data including, for example, acquired data output by the DAS 12, a sinogram generated based on the acquired data, a reconstructed image (PET image) generated based on the sinogram, and the like. The memory 41 also stores a trained model M described below.

The sinogram is data that represents the radiation dose indicated by each acquired data output by the DAS 12 for each position and angle within the radiation detector 11 where the PET detection elements 110 are disposed. For example, when there is a lesion inside the body of the subject P, the radiopharmaceutical gathers at a position of the lesion and more radiation is detected. Thus, the position of the lesion inside the body of the subject P can be identified from the radiation dose indicated by the sinogram. A PET image is an image that visualizes the radiation dose at each position indicated by the sinogram so that a person who performs a PET examination can visually recognize the lesion.

These data may be stored in an external memory with which the PET apparatus 1 can communicate, instead of (or in addition to) the memory 41. The external memory is controlled by a cloud server that manages the external memory by, for example, receiving read and write requests. The external memory is realized by, for example, a system referred to as a picture archiving and communication system (PACS). The PACS is a medical image management system that systematically stores images and the like captured by various imaging diagnostic apparatuses.

The display 42 displays various types of information. For example, the display 42 displays an image (for example, whole body image) generated by the processing circuitry 50, a graphical user interface (GUI) image or the like for receiving various operations performed by an operator (doctor, technician, or the like) of the PET apparatus 1, and the like. The display 42 is, for example, a liquid crystal display (LCD), a cathode ray tube (CRT) display, an organic electroluminescence (EL) display, or the like. The display 42 may be included in the gantry apparatus 10. The display 42 may be a desktop type or a display apparatus (for example, a tablet terminal) that is capable of wirelessly communicating with a main body of the console apparatus 40.

The input interface 43 receives various input operations performed by the operator of the PET apparatus 1 and outputs an electrical signal indicating the details of the received input operation to the processing circuitry 50. For example, the input interface 43 receives input operations for a collection condition for collecting scan data, a generation condition for generating a sinogram, a reconstruction condition for reconstructing a PET image, an image processing condition for generating a post-processing image from the PET image, and the like. The input interface 43 includes, for example, a mouse, a keyboard, a trackball, a switch, a button, a joystick, a touch panel, and the like. The input interface 43 may be, for example, a user interface that receives an audio input from a microphone or the like. In a case where the input interface 43 is a touch panel, the input interface 43 may also have a display function of the display 42.

In the present specification, the input interface 43 is not limited to those equipped with physical operating components, such as a mouse and a keyboard. For example, an example of the input interface 43 also includes electrical signal processing circuitry that receives an electrical signal corresponding to an input operation from an external input device provided separately from the apparatus and outputs the electrical signal to control circuitry.

The network connection circuitry 44 includes, for example, a network card including a printed circuit board, a wireless communication module, or the like. The network connection circuitry 44 implements an information communication protocol that corresponds to a form of a network to be connected. The network includes, for example, a local area network (LAN), a wide area network (WAN), the Internet, a cellular network, a dedicated line, and the like.

The processing circuitry 50 controls the overall operation of the PET apparatus 1. The processing circuitry 50 executes, for example, a system control function 51, an acquisition function 52, an estimation function 53, a reconstructed image generation function 54, a display control function 55, a training function 56, and the like. The processing circuitry 50 realizes these functions by, for example, a hardware processor executing a program (software) stored in the memory 41.

The term "hardware processor" refers to types of circuitry, such as a CPU, a graphics processing unit (GPU), an application specific integrated circuit (ASIC), a programmable logic device (for example, a simple programmable logic device (SPLD) or a complex programmable logic device (CPLD), and a field programmable gate array (FPGA)), and the like. Instead of storing a program in the memory 41, the hardware processor may be configured so that the program is directly incorporated into circuitry of the hardware processor. In this case, the hardware processor realizes the functions by reading and executing the program incorporated into the circuitry. The hardware processor is not limited to being configured as single circuitry, but may be configured as a single hardware processor by combining a plurality of independent circuitry components to realize each function. Further, a plurality of components may be integrated into a single hardware processor to realize each function. Furthermore, a plurality of components may be integrated into a single dedicated large scale integration (LSI) to realize each function. Here, the program (software) may be stored in advance in a storage device (storage device provided with a non-transitory storage medium) that configures the memory 41, such as ROM, RAM, HDD, flash memory, or the like, or may be stored in a removable storage medium (non-transitory storage medium) such as a digital versatile disk (DVD), a compact disc (CD)-ROM, or the like, and installed in a storage device provided in the console apparatus 40 by inserting the storage medium into a drive device provided in the console apparatus 40. Further, the program (software) may be downloaded in advance from another computer apparatus via a network to which the network connection circuitry 44 is connected, and installed into a storage device provided in the console apparatus 40.

Each of the components included in the console apparatus 40 or the processing circuitry 50 may be distributed and realized by a plurality of hardware components. The processing circuitry 50 may be realized by a processing apparatus capable of communicating with the console apparatus 40, rather than being provided in the console apparatus 40. The processing apparatus is, for example, a workstation that is connected to one PET apparatus, or an apparatus (for example, a cloud server) that is connected to a plurality of PET apparatuses and collectively executes processing equivalent to that of the processing circuitry 50 described below. In other words, the configuration according to the present embodiment can also be realized as a PET examination system in which a PET apparatus and such a processing apparatus are connected via a network. In this case, the processing apparatus, such as a workstation, is an example of a "medical information processing apparatus" as described in the claims.

The system control function 51 controls various functions of the processing circuitry 50 based on, for example, an input operation received by the input interface 43. The system control function 51 also controls various functions of the gantry apparatus 10 by issuing instructions to the DAS 12, the control apparatus 14, and the couch driving apparatus 32 based on, for example, an input operation received by the input interface 43. The system control function 51 is an example of a "system control unit" as described in the claims.

The acquisition function 52 acquires a plurality of scan data of radiation based on the radiopharmaceutical administered to the subject P, which has been detected in time series by the radiation detector 11. The acquisition function 52 also acquires a plurality of scan data detected by the radiation detector 11 by repeatedly scanning each body region of the subject P along the body axis direction in time series. The acquisition function 52 is an example of the "acquisition unit" as described in the claims.

The estimation function 53 estimates missing scan data (hereinbelow referred to as "missing scan data") corresponding to a region other than a scan target region of the subject P in each scan, based on a correlation among the plurality of scan data acquired by the acquisition function 52 in the body axis direction or a time axis direction of the time series. The estimation function 53 estimates the missing scan data using the trained model M. The estimation function 53 estimates whole body scan data based on output data that has been acquired by inputting, into the trained model M, the plurality of scan data detected by the radiation detector 11 through repeatedly scanning each region of the subject P in the body axis direction in time series. The estimation function 53 is an example of an "estimation unit" as described in the claims.

The estimation function 53 may estimate the missing scan data based on the plurality of scan data acquired by the acquisition function 52, instead of using the trained model M. The estimation function 53 may estimate the missing scan data by, for example, performing numerical calculation, such as arranging the acquired scan data in chronological order and blending the scan data together.

The reconstructed image generation function 54 generates a sinogram by performing predetermined processing on the acquired data output from the DAS 12, generates a PET image by performing predetermined reconstruction processing, such as a filtered back projection method and an iterative reconstruction method, on the generated sinogram, and stores the generated PET image in the memory 41. The reconstructed image generation function 54 generates time-series PET images of the subject P having a width wider than a width of the radiation detector 11, based on the plurality of scan data acquired by the acquisition function 52. The reconstructed image generation function 54 generates time-series PET images of the whole body of the subject P, based on the plurality of scan data detected by the radiation detector 11 having a width that cannot cover the whole body of the subject P. The reconstructed image generation function 54 is an example of the "reconstructed image generation unit" as described in the claims.

The display control function 55 controls a display mode of the display 42. For example, the display control function 55 controls the display 42 to display a PET image generated by the processing circuitry 50, a GUI image for receiving various operations from the operator of the PET apparatus 1, and the like. The display control function 55 is an example of a "display control unit" as described in the claims.

The training function 56 generates the trained model M that is trained to output whole body scan data corresponding to the whole body of the subject P, including the missing scan data other than the scan target region in each scan in response to input of a plurality of scan data scanned for each body region of the subject P in the body axis direction. The trained model M is generated by training using a technique, such as a convolutional neural network (CNN) and a deep neural network (DNN). The CNN is a neural network in which several layers, such as a convolution layer and a pooling layer, are connected. The DNN is a neural network in which an arbitrary form layer is connected to a plurality of layers. The trained model M may be generated using arbitrary machine learning technique, such as gradient methods including stochastic gradient descent (SGD), Momentum SGD, adaptive gradient algorithm (AdaGrad), root mean square propagation (RMSProp), adaptive delta (AdaDelta), adaptive moment estimation (Adam) and the like, a logistic regression analysis, a technique based on a support vector machine, and the like. The training function 56 is an example of a "training unit" as described in the claims.

The trained model M may be trained to output a whole body image corresponding to the whole body of the subject P, including a missing image other than the scan target region in each scan, in response to an input of an image generated based on a plurality of scan data scanned for each body region of the subject P in the body axis direction. Alternatively, the trained model M may be trained to output a whole body sinogram corresponding to the whole body of the subject P, including a missing sinogram other than the scan target region in each scan, in response to an input of a sinogram generated based on a plurality of scan data scanned for each body region of the subject P in the body axis direction.

### Generation Processing for Whole Body Image

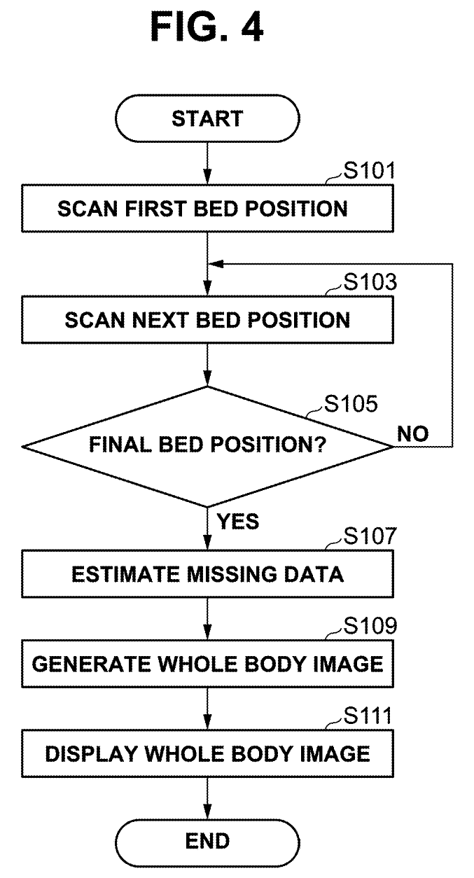

Next, a procedure of processing for generating a whole body image by the PET apparatus 1 is described. FIG. 4 is a flowchart illustrating an example of processing for generating a whole body image by the PET apparatus 1 according to the embodiment. The processing illustrated in FIG. 4 is started in a state where an operator completes setting of an imaging condition (imaging protocol) via the input interface 43 and the subject P as an examination target is placed on the couch apparatus 30. A data collection method by the processing illustrated in FIG. 4 may be either a step-and-shoot method or a continuous method, in which the couch apparatus 30 moves continuously while data is being collected. The following description is an example case using the step-and-shoot method.

First, in step S101, the system control function 51 controls the gantry apparatus 10 based on, for example, an input operation (scan start operation) performed by the operator and received via the input interface 43 to execute a scan of a first bed position (scan 1, for example, a head region scan) of the subject P. Accordingly, the acquisition function 52 acquires scan data corresponding to the scan 1 and stores the acquired scan data in the memory 41.

Next, in step S103, the system control function 51 controls the gantry apparatus 10 to execute a scan of the next bed position (scan 2, for example, a chest region scan) of the subject P. Accordingly, the acquisition function 52 acquires scan data corresponding to the scan 2 and stores the acquired scan data in the memory 41.

Next, in step S105, the system control function 51 determines whether a scan of a final bed position is completed based on the imaging condition (imaging protocol) set in advance. In a case where the system control function 51 determines that the scan of the final bed position is not completed (NO in step S105), the processing returns to step S103 described above, and similar processing is repeated.

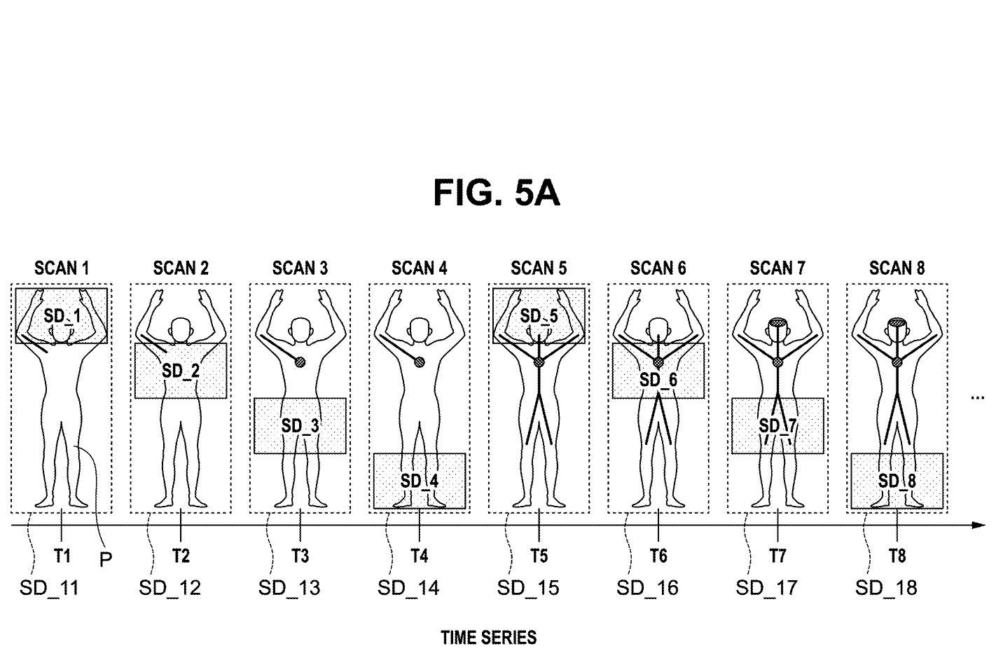

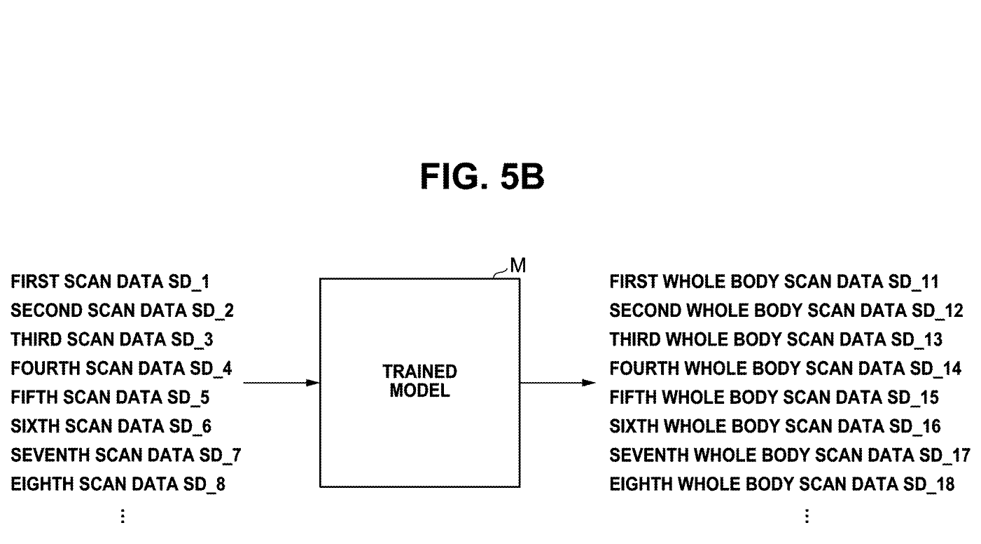

FIG. 5A illustrates a procedure of scan processing executed by the PET apparatus 1 according to the embodiment. In the scan 1 (first bed position) executed at the time T1, first scan data SD_1 of the head region of the subject P is acquired. Next, in the scan 2 (second bed position) executed at the time T2, second scan data SD_2 of the chest region of the subject P is acquired. Next, in the scan 3 (third bed position) executed at the time T3, third scan data SD_3 of the lower abdomen region of the subject P is acquired. Next, in the scan 4 (fourth bed position) executed at the time T4, fourth scan data SD_4 of the leg region of the subject P is acquired. Subsequently, a set of four scans to acquire fifth scan data SD_5 (the head region), sixth scan data SD_6 (the chest region), seventh scan data SD_7 (the lower abdomen region), and eighth scan data SD_8 (the leg region), is repeatedly executed. The series of scans may be executed in one direction from the head region to the leg region as described above, or may be executed in a reciprocating manner, for example, from the head region to the leg region and then from the leg region to the head region.

Returning to FIG. 4, in step S105, in a case where the system control function 51 determines that the scan of the final bed position is completed (YES in step S105), in step S107, the estimation function 53 estimates missing scan data corresponding to the region other than the scan target region of the subject P in each scan, based on the plurality of scan data acquired by the acquisition function 52. The estimation function 53 inputs the scan data, which has been detected via the radiation detector 11 by repeatedly scanning each body region of the subject P in the body axis direction in time series, into the trained model M to estimate the whole body scan data.

FIG. 5B is a diagram illustrating input and output data of the trained model M according to the embodiment. The input data is the scan data (first scan data SD_1 to eighth scan data SD_8, ...) acquired by scanning each body region of the subject P in steps S101 and S103 as described above. The output data is the whole body scan data corresponding to each scan executed in steps S101 and S103 as described above. As illustrated in FIG. 5A, the output data is, for example, first whole body scan data SD_11 for the scan 1. The first whole body scan data SD_11 includes the first scan data SD_1, which is actual scan data of the head region of the subject P acquired by actually scanning, and estimated scan data of the region other than the head region of the subject P.

Returning to FIG. 4, next, in step S109, the reconstructed image generation function 54 generates a whole body image of the subject P using the whole body scan data generated by the estimation function 53.

Next, in step S111, the display control function 55 causes the display 42 to display the whole body image of the subject P generated by the reconstructed image generation function 54. Thus, the operator can observe the whole body image of each scan displayed on the display 42. Then, the processing in the present flowchart is terminated.

The image to be displayed on the display 42 is not limited to the whole body image of the subject P. For example, lower limb data (leg region and the like) of the subject P may be unimportant depending on pathology and thus may not need to be output. In such a case, the display control function 55 may cause the display 42 to display an image obtained by excluding an unimportant region of the subject P from the whole body image of the subject P generated by the reconstructed image generation function 54. Alternatively, the estimation function 53 may exclude unimportant data of the subject P (for example, lower limb scan data) from an estimation target. For example, unimportant data of the subject P may be excluded from the output data of the trained model M. Alternatively, the reconstructed image generation function 54 may generate an image in which the unimportant region of the subject P is excluded using the whole body scan data generated by the estimation function 53.

### Training Processing

Next, training processing by the training function 56 is described. The training function 56 uses, as training data, whole body scan data acquired using a conventional total body PET apparatus equipped with a detector that can cover a whole body. The training function 56 extracts scan data for each body region of the subject P (hereinbelow, referred to as "extracted scan data") from the whole body scan data and uses a pair of the extracted scan data and the whole body scan data, which is the extraction source, as the training data.

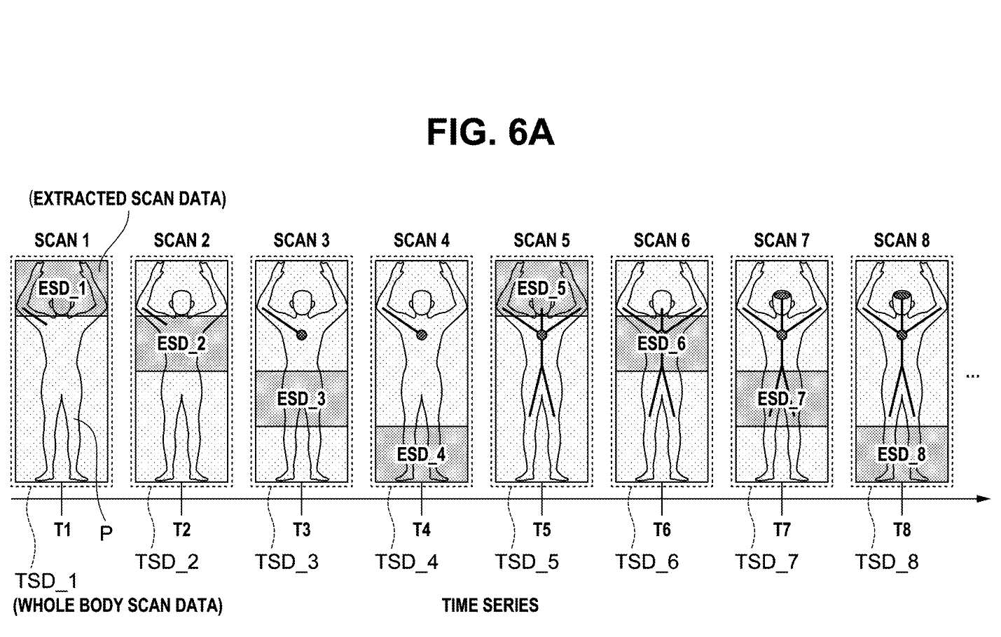

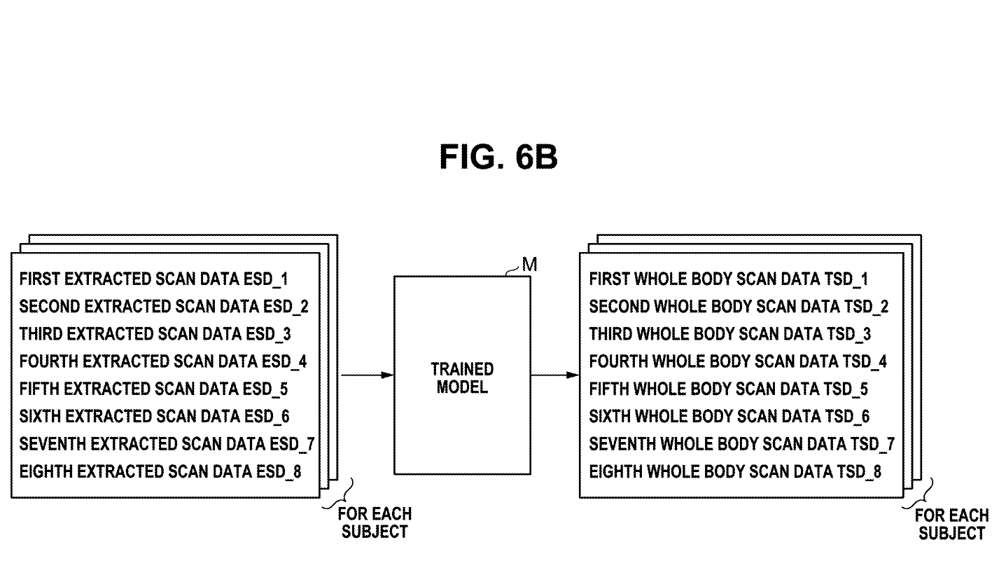

FIG. 6A is a diagram illustrating training data prepared based on the whole body scan data captured by the total body PET apparatus. As illustrated in FIG. 6A, for example, first whole body scan data TSD_1 to eighth whole body scan data TSD_8 of the subject P captured by the total body PET apparatus at the times T1 to T8, respectively, are prepared. Then, first extracted scan data ESD_1 to eighth extracted scan data ESD_8 for each body region of the subject P are extracted from these first whole body scan data TSD_1 to eighth whole body scan data TSD_8, respectively, and the pair of the extracted scan data and the whole body scan data of the extraction source is used as the training data. Preprocessing as described above is performed on whole body scan data acquired by scanning a plurality of subjects, and a large amount of training data is prepared.

FIG. 6B is a diagram illustrating input and output data of the trained model M in a training phase according to the embodiment. The input data is the extracted scan data (for example, first extracted scan data ESD_1 to eighth extracted scan data ESD_8) for each subject. Each of the extracted scan data is associated with information about a scan time. Alternatively, in a case where the first whole body scan data TSD_1 to eighth whole body scan data TSD_8 have been acquired by corresponding to scan conditions (scan interval and the like) at the time of actual examination of the subject by the PET apparatus 1 according to the present embodiment, the extracted scan data may not be associated with the information about the scan time. The output data is the whole body scan data (for example, first whole body scan data TSD_1 to eighth whole body scan data TSD_8) for each subject. The training function 56 performs training processing on the trained model M using the training data (a pair of the input data and the output data) prepared as described above.

In other words, the training function 56 generates the trained model M by learning the training data including a pair of the whole body scan data detected by the detector that can cover the whole body of the subject P and the extracted scan data extracted from the whole body scan data for each body region of the subject P.

According to the above-described first embodiment, it is possible to generate time-series PET images of a subject having a width wider than a width of a detector. Particularly, even in a case where there is no detector that can cover a whole body, time-series whole body images can be generated. Thus, a cost of the detector can be reduced. In addition, a large-scale apparatus, such as a total body PET apparatus, becomes unnecessary, and time and effort required for maintenance can be reduced.

### Second Embodiment

Next, a second embodiment is described. The second embodiment is different from the first embodiment in that a scan time for each body region of a subject is variable in consideration of a body region that is important in terms of biological information. In the following description, configurations and functions that are similar to those according to the first embodiment are denoted by the same reference numerals in the first embodiment, and the redundant detailed descriptions are omitted.

The system control function 51 controls a scan so that a scan time for a body region of a subject that is important in terms of biological information is extended. For example, a collection time is extended in scanning a region, such as a region that is important in terms of blood flow of the heart or the like, a region where a target pathology is likely to occur, or the like, so that a whole body image can be generated more accurately. Similarly, when a drug reaches the heart at a certain period of time from a start of drug injection, data at that time is likely to be important. A region with large time-series changes is identified based on research results of total body PET equipped with a detector that covers a whole body, and a collection time for a specific region at a certain period of time is considered important, and the scan time may be extended.

An important region may be set based on an input operation performed by an operator received via the input interface 43. Alternatively, the important region may be automatically set according to an imaging condition (imaging protocol). An example of control to extend the scan time is to increase the number of scans corresponding to the important region.

Further, in the training phase of the trained model M by the training function 56, accuracy of estimating the important region may be improved by including a large amount of data of a body region that is important in terms of biological information on the subject in the training data. Accordingly, it is possible to generate a whole body image more accurately.

For data other than the important region, a whole body image may be output as reference data. In other words, since the collection time is relatively short for an unimportant part, this part may be treated and explicitly displayed as the reference data (for example, displaying a note such as "this part is the reference data" or the like). Accordingly, the operator can focus on observing data of the important region while treating the other part, which is the unimportant part, as reference, whereby a workload of image diagnosis can be reduced.

In other words, the system control function 51 executes a scan under the condition in which the collection time of scan data for each body region of the subject in the body axis direction is variable based on biological information of the subject. Further, when displaying time-series PET images of the whole body of the subject generated based on the estimated missing scan data, the display control function 55 displays an unimportant region of the subject's body as the reference data.

### Third Embodiment

Next, a third embodiment is described. The third embodiment is different from the second embodiment in that importance of extending a scan time is determined not based on a biological information approach, but by factoring in a region (such as the heart) having features that have a large influence on results (output data) during the training of the trained model M. In the following description, configurations and functions that are similar to those according to the first and second embodiments are denoted by the same reference numerals in the first and second embodiments, and the redundant detailed descriptions are omitted.

In the training phase of the trained model M by the training function 56, accuracy of training is improved by including, in the training data, a large amount of scan data corresponding to a region (describing a case of the heart, or the like) having features that have a large influence on the estimation results (output data) of the trained model M (by extending a scan time of the region). The estimation function 53 can improve the estimation accuracy using the trained model M that is trained using the training data prepared in this way. The scan time of the important region may be extended in the same manner in actually scanning the subject P. Alternatively, no particular data may be generated during the training, and the scan time of the important region may be extended only during actual scanning.

For data other than the important region, a whole body image may be output as reference data. In other words, since the collection time is relatively short for an unimportant part, this part may be treated and explicitly displayed as the reference data (for example, displaying a note such as "this part is the reference data" or the like). Accordingly, the operator can focus on observing data of the important region while treating the other unimportant part as reference, whereby a workload of image diagnosis is reduced.

In other words, the system control function 51 executes a scan under the condition in which the collection time of scan data for each body region of the subject in the body axis direction is variable based on magnitude of the influence on the output of the trained model M. Further, when displaying time-series PET images of the whole body of the subject generated based on the estimated missing scan data, the display control function 55 displays an unimportant region of the subject's body as the reference data.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus configured to process scan data acquired by a plurality of detectors detecting gamma ray emitted from a subject mounted on a couchtop, the plurality of detectors being supported by a frame included in a positron emission tomography (PET) apparatus, at least one of the couchtop or the frame being configured to move in a longitudinal direction of the couchtop, the medical image processing apparatus comprising:
processing circuitry configured to:
acquire a first plurality of scan data corresponding to a first region of the subject in the longitudinal direction and a second plurality of scan data corresponding to a second region of the subject in the longitudinal direction, the first plurality of scan data being acquired in a first period by the plurality of detectors in a state where the plurality of detectors is located at a position corresponding to the first region, the second plurality of scan data being acquired in a second period after the first period by the plurality of detectors in a state where the plurality of detectors is located at a position corresponding to the second region;
estimate scan data corresponding to a region of the subject different from the first region in the first period based on the first plurality of scan data and the second plurality of scan data; and
generate a PET image corresponding to the region of the subject different from the first region in the first period based on the estimated scan data.

2. The medical image processing apparatus according to claim 1, wherein the processing circuitry is configured to estimate the scan data corresponding to the region of the subject different from the first region in the first period using a trained model.

3. The medical image processing apparatus according to claim 1, wherein the processing circuitry is further configured to set a length of the first period and a length of the second period based on biological information of the subject.

4. The medical image processing apparatus according to claim 1, wherein the processing circuitry is further configured to set a length of the first period and a length of the second period based on a condition of the scanning of the subject.

5. The medical image processing apparatus according to claim 1, wherein the processing circuitry is further configured to set a length of the first period and a length of the second period based on an instruction by a user of the PET apparatus.

6. A positron emission tomography (PET) apparatus comprising:
a couchtop on which a subject to be scanned is mounted;
a plurality of detectors configured to detect gamma ray emitted from the subject mounted on the couchtop;
a frame configured to support the plurality of detectors, at least one of the couchtop or the frame being configured to move in a longitudinal direction of the couchtop: and
processing circuitry configured to:
acquire a first plurality of scan data corresponding to a first region of the subject in the longitudinal direction and a second plurality of scan data corresponding to a second region of the subject in the longitudinal direction, the first plurality of scan data being acquired in a first period by the plurality of detectors in a state where the plurality of detectors is located at a position corresponding to the first region, the second plurality of scan data being acquired in a second period after the first period by the plurality of detectors in a state where the plurality of detectors is located at a position corresponding to the second region;
estimate scan data corresponding to a region of the subject different from the first region in the first period based on the first plurality of scan data and the second plurality of scan data; and
generate a PET image corresponding to the region of the subject different from the first region in the first period based on the estimated scan data.

7. The PET apparatus according to claim 6, wherein the processing circuitry is configured to estimate the scan data corresponding to the region of the subject different from the first region in the first period using a trained model.

8. The PET apparatus according to claim 6, wherein the processing circuitry is further configured to set a length of the first period and a length of the second period based on biological information of the subject.

9. The medical image processing apparatus according to claim 6, wherein the processing circuitry is further configured to set a length of the first period and a length of the second period based on a condition of the scanning of the subject.

10. The medical image processing apparatus according to claim 6, wherein the processing circuitry is further configured to set a length of the first period and a length of the second period based on an instruction by a user of the PET apparatus.

11. A medical image processing method to process scan data acquired by a plurality of detectors detecting gamma ray emitted from a subject mounted on a couchtop, the plurality of detectors being supported by a frame included in a positron emission tomography (PET) apparatus, at least one of the couchtop or the frame being configured to move in a longitudinal direction of the couchtop, the method comprising:
acquiring a first plurality of scan data corresponding to a first region of the subject in the longitudinal direction and a second plurality of scan data corresponding to a second region of the subject in the longitudinal direction, the first plurality of scan data being acquired in a first period by the plurality of detectors in a state where the plurality of detectors is located at a position corresponding to the first region, the second plurality of scan data being acquired in a second period after the first period by the plurality of detectors in a state where the plurality of detectors is located at a position corresponding to the second region;
estimating scan data corresponding to a region of the subject different from the first region in the first period based on the first plurality of scan data and the second plurality of scan data; and
generating a PET image corresponding to the region of the subject different from the first region in the first period based on the estimated scan data.
